(19) United States
(12) Patent Application Publication
(10) Pub. No.: US 2026/0356694 A1
(43) Pub. Date: Nov. 12, 2026

(54) WATER DISPENSING APPARATUS

(71) Applicant: LG ELECTRONICS INC., Seoul (KR)

(72) Inventors: Sangjoon LEE, Seoul (KR); Kowoon BAEK, Seoul (KR); Yuri CHOI, Seoul (KR); Minyoung PARK, Seoul (KR)

(21) Appl. No.: 19/109,246

(22) PCT Filed: Sep. 7, 2023
(86) PCT No.: PCT/KR2023/013382

§ 371 Date: Mar. 6, 2025

(30) Foreign Application Priority Data
Sep. 8, 2022 (KR) 10-2022-0113961

(51) Int. Cl. C02F 1/00; A61L 2/10; A61L 2/24; B67D 1/00; B67D 1/08; G01N 33/18

(52) U.S. Cl. CPC C02F 1/001; A61L 2/10; A61L 2/24; B67D 1/0003; B67D 1/0884; B67D 1/0888; B67D 1/0895; C02F 1/008; G01N 33/1893; A61L 2202/14; B67D 2001/0093; B67D 2210/0001; B67D 2210/00015; C02F 2201/005; C02F 2209/11; C02F 2307/10

(57) ABSTRACT

A water dispensing apparatus, according to one embodiment of the present disclosure, includes: a water supply flow path having flowing therein raw water supplied from a water supply source; a filter for generating purified water by filtering the raw water supplied to the water supply flow path; a purified water flow path having flowing therein the purified water which has passed through the filter; a sensing flow path branching off from the water supply flow path and having the raw water flowing therein; and a water quality measuring unit connected to the purified water flow path and the sensing flow path so as to have the purified water or the raw water entering therein, wherein the water quality measuring unit measures the water quality of the purified water when the purified water enters through the purified water flow path, and measures the water quality of the raw water when the raw water enters through the sensing flow path.

### TECHNICAL FIELD

The present disclosure relates to a water dispensing apparatus and an operating method thereof, and more particularly, to a water dispensing apparatus including a sensor capable of discriminating the quality of raw water and outlet water, and an operating method thereof.

### BACKGROUND ART

A water dispensing apparatus supplies water and may take out a desired amount of water at a desired temperature according to the manipulation of a user. The water dispensing apparatus as such may be applied to various fields, but may be applied representatively to a refrigerator and a water purifier. In particular, the water dispensing apparatus provided in the refrigerator and the water purifier is configured to have a function that enables supplying of a preset amount of water according to the manipulation of a user. Recently, for these water dispensing apparatuses, the water dispensing apparatuses that may supply not only purified water but also cold and hot water have been developed.

For example, a water purifier is connected to a water supply source such as a tap to receive raw water, uses a filter to remove floating matters or harmful substances contained in the raw water, and is configured to purify and take out a desired amount of water according to the manipulation of a user. A variety of water purifier products are being released that may purify water as well as heat or cool the purified water to supply cold or hot water. In addition, recently, water purifiers that are small in size and may be installed in various installation environments are being developed.

In a case where the water dispensing apparatus is used for a long time, microorganisms or the like may be propagated in or contaminate pipes, valves, or water outlets. In addition, due to the elapse of the replacement cycle of a filter, floating matters or harmful substances contained in raw water may not be removed. Accordingly, it is important for the water dispensing apparatus to accurately measure water quality, perform hygienical management, and also manage water purification quality performance.

The related art of Korean Patent Application Publication No. 10-2017-0005264 discloses a water treatment apparatus including: a water quality measuring part which measures the quality of water stored in the purification tank and generates information about the water quality; a circulation channel which provides a path resupplying the stored purified water in the purification tank to the filtration part; and a controlling part which drains the stored purified water in the purification tank depending on the water quality information or resupplies the water to the filtration part through the circulation channel. This water treatment apparatus is a system that may measure only purified water quality and is unable to sense filter defects or contamination of raw water.

### DISCLOSURE

### Technical Problem

An aspect of the present disclosure is directed to providing a water dispensing apparatus capable of measuring raw water quality and responding to water quality accidents caused by raw water.

Another aspect of the present disclosure is directed to providing a water dispensing apparatus capable of managing and visualizing the difference in water quality measurement values between raw water and water purifier outlet water.

In the case of tap water and purified outlet water, the water quality contamination level is normally low. In order to measure a low-concentration contamination level, it is important to minimize the occurrence of deviation in the measurement values. An aspect of the present disclosure is directed to providing a system capable of measuring the water qualities of both raw water and outlet water using a single water quality sensor and fundamentally removing deviations that may occur between sensors when sensors to each of the raw water and outlet water are applied.

Yet another aspect of the present disclosure is directed to providing a water dispensing apparatus capable of reducing material costs by sensor publicization and enabling a compact product configuration.

Yet another aspect of the present disclosure is directed to providing a water dispensing apparatus capable of improving sensing accuracy and efficiency by a water pipe configuration and a rinse operation for sensor publicization.

Yet another aspect of the present disclosure is directed to providing a water dispensing apparatus capable of easily determining and managing a contamination level.

### Technical Solution

A water dispensing apparatus according to embodiments of the present disclosure may prevent sensing errors and manage water quality using the same standard by measuring the water qualities of raw water and purified water in a common water quality measuring unit.

The water dispensing apparatus according to embodiments of the present disclosure may appropriately respond to water quality accidents caused by raw water and deterioration of purified water quality by acquiring specific water quality data of raw water and purified water with one sensor and analyzing and comparing the results.

The water dispensing apparatus according to embodiments of the present disclosure may improve sensing accuracy and efficiency by configuring a water pipe flow path for sensor publicization and a control logic such as a rinse operation.

The water dispensing apparatus according to an embodiment of the present disclosure includes: a water supply flow path having flowing therein raw water supplied from a water supply source; a filter for generating purified water by filtering the raw water supplied to the water supply flow path; a purified water flow path having flowing therein the purified water which has passed through the filter; a sensing flow path branching off from the water supply flow path and having the raw water flowing therein; and a water quality measuring unit connected to the purified water flow path and the sensing flow path so as to have the purified water or the raw water entering therein, wherein the water quality measuring unit measures the water quality of the purified water when the purified water enters through the purified water flow path, and measures the water quality of the raw water when the raw water enters through the sensing flow path.

When the water quality of the raw water is measured, the water quality measuring unit may measure the water quality of the purified water after performing a rinse operation in which the purified water passes through at least once.

The water dispensing apparatus according to an embodiment of the present disclosure may further include a water supply valve for opening and closing the purified water flow path, and a sensing valve for opening and closing the sensing flow path.

The water supply flow path includes a first water supply flow path connecting the water supply source and the water supply valve, and a second water supply flow path connecting the water supply valve and the filter, wherein one end of the sensing flow path may be connected to the first water supply flow path, and the other end thereof may be connected to the water quality measuring unit.

The water dispensing apparatus according to an embodiment of the present disclosure may further include a three-way valve for selectively supplying the raw water to the purified water flow path and the sensing flow path.

The water quality measuring unit may include a turbidity sensor that irradiates light to the raw water or a portion of the purified water and senses turbidity based on a pattern of scattered light received.

The water quality measuring unit may include at least one of a turbidity sensor, an ion sensor, a chlorine sensor, a total dissolved solids (TDS) sensor, or a BOD (biochemical oxygen demand) sensor, and may measure at least one of turbidity, pH, residual chlorine, TDS, or dissolved oxygen of entering water.

The water dispensing apparatus according to an embodiment of the present disclosure may further include: a water outlet through which the purified water is discharged; a water outlet flow path guiding the purified water to the water outlet; a drainage flow path branched off from the water outlet flow path between the water quality measuring unit and the water outlet through which the raw water or the purified water is drained; and a water outlet valve selectively supplying the raw water or the purified water to the water outlet flow path and the drainage flow path.

The water dispensing apparatus according to an embodiment of the present disclosure may further include: a hot water flow path branched off from the purified water flow path at one side; a hot water module provided in the hot water flow path to heat purified water passing through the hot water flow path; a cold water flow path branched off from the purified water flow path at one side; and a cooling module provided in the cold water flow path to cool purified water passing through the cold water flow path.

The hot water flow path and the cold water flow path may be joined to the water outlet flow path or joined again to the purified water flow path.

The water dispensing apparatus according to an embodiment of the present disclosure may further include a water outlet sterilization module that irradiates ultraviolet rays to the water outlet.

The water dispensing apparatus according to an embodiment of the present disclosure may further include a sterilization flow path having one side branched off from the water supply flow path and the other side connected to the filter, and a sterilization module provided in the sterilization flow path to heat water passing through the sterilization flow path.

The water dispensing apparatus according to an embodiment of the present disclosure may further include a switching valve for supplying the raw water to the water supply flow path or the sterilization flow path, and a sensing valve for opening and closing the sensing flow path.

The water dispensing apparatus according to an embodiment of the present disclosure may further a control unit that controls to perform a feedback operation based on water quality data measured by the water quality measuring unit.

When the water quality data of the raw water is not outside a preset normal range, the control unit may control to perform a rinse operation in which the purified water passes through the water quality measuring unit at least once.

The control unit may perform the rinse operation again and measure the water quality of the purified water again when the water quality data of the purified water measured after the rinse operation differs by a reference value or more from the water quality data of the purified water measured before the rinse operation.

The water dispensing apparatus according to an embodiment of the present disclosure may further include an output unit that displays raw water quality abnormality information when the water quality data of the raw water is outside a preset normal range, and displays purified water quality abnormality information when the water quality data of the purified water is outside a preset normal range.

When there is an abnormality in the water quality data of the purified water, after measuring the water quality of the raw water, the control unit may control the output unit to output raw water quality abnormality notification information when there is an abnormality in the water quality data of the raw water, and may control the output unit to output purified water quality abnormality notification information when there is no abnormality in the water quality data of the raw water.

The water dispensing apparatus according to an embodiment of the present disclosure may further include a water outlet through which the purified water is discharged and a water outlet sterilization module that irradiates ultraviolet rays to the water outlet, wherein the control unit may operate the water outlet sterilization module for a predetermined time based on the water quality data measured by the water quality measuring unit.

The water dispensing apparatus according to an embodiment of the present disclosure may further include a sterilization flow path having one side branched off from the water supply flow path and the other side connected to the filter, and a sterilization module provided in the sterilization flow path to heat water passing through the sterilization flow path, wherein the control unit may operate the sterilization module for a predetermined time based on the water quality data measured by the water quality measuring unit.

### Advantageous Effects

According to at least one of the embodiments of the present disclosure, by measuring the water qualities of raw water and purified water with one sensor, it is possible to respond to water quality accidents caused by the raw water and deterioration of purified water quality.

According to at least one of the embodiments of the present disclosure, the sensing accuracy and efficiency can be improved by a water pipe configuration and a rinse operation for sensor publicization.

According to at least one of the embodiments of the present disclosure, the contamination level can be easily determined and managed.

According to at least one of the embodiments of the present disclosure, by publicization of sensors of a same type within a water dispensing apparatus, additional material costs can be reduced and a compact product configuration is possible.

Various other benefits will be described directly or implicitly in the detailed description according to the embodiments of the present disclosure to be described later.

### DESCRIPTION OF DRAWINGS

FIG. 1 is a conceptual diagram illustrating water quality sensing of a water dispensing apparatus according to an embodiment of the present disclosure.

FIG. 2 is a block diagram illustrating the main configuration of a water dispensing apparatus according to an embodiment of the present disclosure.

FIG. 3 is a flowchart of an operation method of a water dispensing apparatus according to an embodiment of the present disclosure.

FIG. 4 is a flowchart of an operation method of a water dispensing apparatus according to an embodiment of the present disclosure.

FIG. 5 is a flowchart of an operation method of a water dispensing apparatus according to an embodiment of the present disclosure.

FIG. 6 is a conceptual diagram illustrating a water dispensing apparatus according to an embodiment of the present disclosure.

FIGS. 7 to 9 are diagrams for reference in describing the operation of the water dispensing apparatus of FIG. 6.

FIG. 10 is a conceptual diagram illustrating a water dispensing apparatus according to an embodiment of the present disclosure.

FIG. 11 and FIG. 12 are diagrams for reference in describing the operation of the water dispensing apparatus of FIG. 10.

### MODE FOR DISCLOSURE

Hereinafter, the exemplary embodiments of the present disclosure are described in detail with reference to the accompanying drawings. The present disclosure may, however, be embodied in many different forms and should not be construed as being limited to the embodiments set forth herein.

In order to clearly and briefly describe the present disclosure, the parts irrelevant to the description are omitted in the drawings. The same reference numerals are used to designate the same or similar parts throughout the specification.

With respect to constituents used in the following description, the suffixes “module” and “unit” are merely given in consideration of only facilitation of description and do not have any special importance or role. Accordingly, the “module” and the “unit” may be used interchangeably.

In addition, it will be understood that, although the terms “first”, “second”, etc. may be used herein to describe various elements, these elements should not be limited by these terms. These terms are only used to distinguish one element from another element.

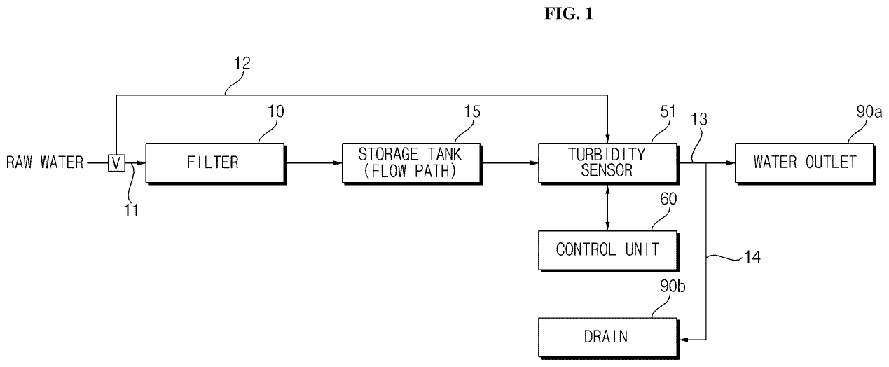

FIG. 1 is a conceptual diagram illustrating water quality sensing of a water dispensing apparatus according to an embodiment of the present disclosure.

Referring to FIG. 1, the water dispensing apparatus according to embodiments of the present disclosure includes a water supply flow path 11 through which raw water supplied from a water supply source flows, and a filter 10 that filters the raw water supplied to the water supply flow path 11 to generate purified water.

The filter 10 is for purification of supplied raw water and filters out various impurities and harmful substances contained in the raw water. One or more filters 10 may be provided, and in a case where a plurality of filters are provided, the filter 10 may be configured to combine filters with various functions. For example, the filter 10 may be provided in three pieces, and may include a pre-carbon filter and a post-carbon filter, and a membrane filter or a hollow fiber membrane filter disposed between the pre-carbon filter and the post-carbon filter. Alternatively, the filter 10 may be configured of a pre-carbon filter and a UF composite filter.

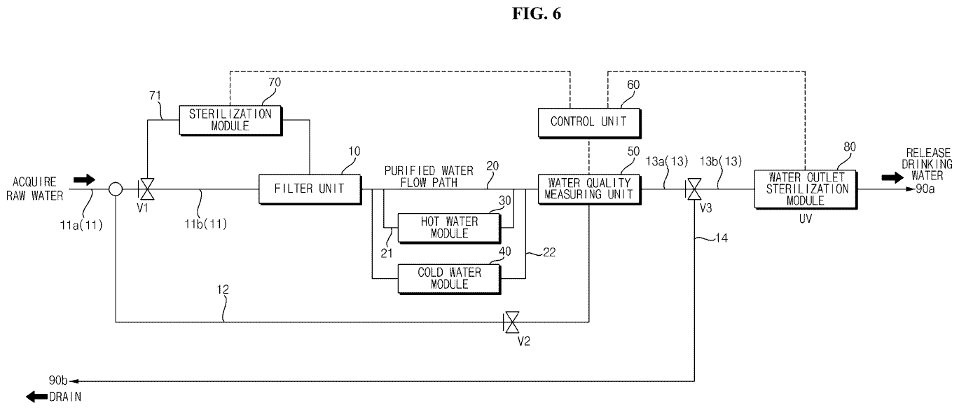

The purified water purified from the filter 10 flows to a storage tank or a flow path 15 (20 of FIG. 6). Since water stored in the storage tank becomes a better environment for microbial growth over time, it is more desirable to flow directly through the flow path 20. The purified water that has passed through the filter 10 flows into the purified water flow path 20.

The water dispensing apparatus includes a sensing flow path 12 branched off from the water supply flow path 11 and through which the raw water flows. The sensing flow path 12 is a dedicated flow path for measuring the water quality of raw water and maybe branched off from a front end of the filter 10 so that the raw water bypasses the filter 10.

A common sensor 51 is connected to the purified water flow path 11 and the sensing flow path 12, so that the water qualities of the purified water flowing through the purified water flow path 11 and the raw water flowing through the sensing flow path 12 may be measured. Accordingly, since one common sensor 51 measures both the water quality of raw water and the water quality of purified water, the sensor cost and the cost of configuring the flow path may be reduced compared to when separate water quality measurement sensor of the raw water and water quality measurement sensor of the purified water are provided. In addition, the deviation between sensors that may occur by using two sensors does not fundamentally occur.

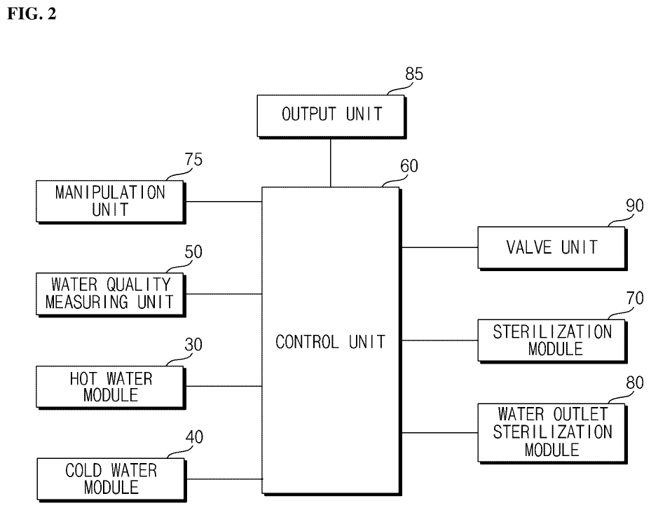

In FIG. 1, a turbidity sensor 51 is exemplified as the common sensor 51. The turbidity sensor 51 measures the turbidity of raw water and purified water, and transmits the sensing data to a control unit 60. The control unit 60 may control other configurations of the water dispensing apparatus based on sensing data from a water quality measuring unit 50 (see FIG. 2) such as the turbidity sensor 51.

The turbidity sensor 51 may sense turbidity (contamination level) through optical sensing. The optical sensing method for measuring turbidity uses a transmitted light method and a scattered light method. The transmitted light method senses turbidity by irradiating light on a fluid, receiving light transmitted through the fluid, and processing data. The scattered light method senses turbidity by receiving scattered light and processing data, and is subdivided according to a method of generating scattered light and a method of processing the data received by light.

The water dispensing apparatus includes the water quality measuring unit 50 having one or more common sensors 51. The water quality measuring unit 50 may include the turbidity sensor 51 that irradiates light to the raw water or a portion of the purified water and senses turbidity based on the scattered light pattern received.

In addition, the water quality measuring unit 50 includes at least one of the turbidity sensor 51, a microorganism detection sensor, a chlorine sensor, a total dissolved solids (TDS) sensor, or a BOD (biochemical oxygen demand) sensor, and may measure at least one of turbidity, microorganisms, residual chlorine, TDS, or dissolved oxygen of entering water. At least one of the sensors provided in the water quality measuring unit 50 may be a common sensor that measures the water qualities of both raw water and purified water.

The water quality measuring unit 50 measures the water quality of the purified water when the purified water enters through the purified water flow path 20, and measures the water quality of the raw water when the raw water enters through the sensing flow path 12.

In this specification, the entering of purified water or raw water in the water quality measuring unit 50 does not mean only that purified water or raw water enters inside the water quality measuring unit 50. For example, a portion of the purified water or raw water may be sampled and discharged after measuring the water quality inside the water quality measuring unit 50. In addition, at least some of the sensors provided in the water quality measuring unit 50 may measure the water quality of a flowing liquid. In this case, the entering of purified water or raw water in the water quality measuring unit 50 may mean that at least a portion of the purified water or raw water passes through the sensible section of the water quality measuring unit 50.

For example, in the case where the turbidity sensor 51 has an internal chamber, the turbidity sensor 51 may measure water quality by irradiating light on the filled water and receiving a scattered light pattern when water enters the internal chamber connected to the flow paths 20, 12 and is filled. In addition, the water in the internal chamber may be discharged after measuring the water quality. Alternatively, a light source unit and a light receiving unit of the turbidity sensor 51 may be disposed in a specific flow path section (for example, a section after the purified water flow path 20 and the sensing flow path 12 are joined) to irradiate light to purified water or raw water passing through the specific flow path section and receive a scattered light pattern.

One or more valves V are disposed in the water supply flow path 11 to selectively supply raw water to the purified water flow paths 15, 20 and the sensing flow path 12. Accordingly, the raw water may flow toward the purified water flow paths 15, 20 or the sensing flow path 12, and the raw water and purified water may be prevented from being supplied to the water quality measuring unit 50 simultaneously.

For example, the valve V may be configured to include a water supply valve that opens and closes the purified water flow paths 15, 20 and a sensing valve that opens and closes the sensing flow path 12. In addition, the valve V may be a three-way valve for selectively supplying the raw water to the purified water flow paths 15, 20 and the sensing flow path 12.

The raw water used in the measurement may be drained toward a drain 90b along a drainage flow path 14. The purified water used in the measurement may be drained to the drain 90b depending on the water quality and priority thereof. For example, when a product is not used for a predetermined time or is turned on, the initially measured purified water may be drained, and the subsequent purified water may be discharged along the water outlet flow path 14 toward a water outlet 90a. In addition, in normal times, purified water purified by the filter 10 may be discharged along the water outlet flow path 14 toward the water outlet 90a.

When the water quality measuring unit 50 measures the water quality of the raw water, the control unit 60 may control the valve V to perform a rinse operation in which the purified water passes through the water quality measuring unit 50 at least once.

The water quality measuring unit 50 may minimize the influence of raw water on the measurement of the water quality of the purified water by measuring the water quality of the purified water after performing the rinse operation. Accordingly, a single water quality sensor of the same type may efficiently and accurately sense both raw water and purified water.

In the case of tap water and purified outlet water, the water quality contamination level is normally low. In order to measure a low-concentration contamination level, it is important to minimize the occurrence of deviation in the measurement values. According to an embodiment of the present disclosure, compared to a technology that measures raw water and outlet water separately, a flow path configuration and control capable of simultaneously measuring raw water and outlet water using one sensor is provided, thereby minimizing an increase in material costs and enabling a compact product configuration.

Since the tap water quality (raw water) and water purifier quality (purified water) are mostly in a low-concentration section, it is most important to minimize measurement deviation between devices by comparison with a single sensor in order to clearly express the difference in performance between raw water and outlet water.

FIG. 2 is a block diagram illustrating the main configuration of a water dispensing apparatus according to an embodiment of the present disclosure.

Referring to FIG. 2, the water dispensing apparatus according to an embodiment of the present disclosure includes the water quality measuring unit 50 and the control unit 60 described above. The water quality measuring unit 50 may utilize water quality measurement sensors such as a turbidity sensor, a microbial sensor, and a TDS sensor to sense water quality contamination of water within a flow path.

In addition, the water dispensing apparatus includes a valve unit 90 having valves for controlling the flow of water. The valve unit 90 may include the valve V described with reference to FIG. 1. In addition, the valve unit 90 may further include a plurality of valves V1, V2, V3 to be described below.

In addition, the water dispensing apparatus includes a hot water module 30 and a cold water module 40 for providing hot and cold water. The hot water module 30 heats purified water and then discharges the same toward the water outlet 90a. The cold water module 40 cools the purified water and then discharges the same toward the water outlet 90a.

In addition, the water dispensing apparatus further includes a manipulation unit 75 and an output unit 85.

The manipulation unit 75 may include one or more buttons for receiving user input. For example, the manipulation unit 75 may be provided with a touch panel and may include a capacity button for selecting a water outlet capacity, a hot water button for selecting hot water and further selecting the temperature of hot water to be released, a purified water button for selecting purified water, a cold water button for selecting cold water, and other function buttons.

The output unit 85 may be provided with a display device such as a display (not shown) or a light emitting diode (LED) (not shown). For example, the output unit 85 may display information such as the driving state of the water dispensing apparatus, the operation state related to the occurrence of an error, or the contamination level of water.

The output unit 85 may be provided with an audio device such as a speaker (not shown) or a buzzer (not shown). For example, the output unit 85 may output a sound effect regarding the driving state of the water dispensing apparatus and may output a predetermined warning sound when an error occurs.

In addition, the water dispensing apparatus may further include modules for hygiene. For example, the water dispensing apparatus may further include a sterilization module 70 using high-temperature water and a water outlet sterilization module 80 for sterilizing the water outlet 90a where contamination is highly likely to occur.

The sterilization module 70 may sterilize bacteria growing in water by instantly heating the water to a high temperature. In addition, the control unit 60 may operate the sterilization module 70 and circulate the sterilizing water (high temperature water) from the sterilization module 70 to another flow path to sterilize the flow path.

The water outlet sterilization module 80 irradiates ultraviolet rays toward the water outlet 90a to remove bacteria or viruses. The water outlet sterilization module 80 may include at least one ultraviolet (UV) lamp or at least one UV light emitting diode (LED).

The water outlet sterilization module 80 may be driven periodically under the control of the control unit 60. In addition, the water outlet sterilization module 80 may be driven for a predetermined period of time before water outlet. More preferably, the control unit 60 may improve efficiency by driving the water outlet sterilization module 80 only when necessary based on water quality data measured by the water quality measuring unit 50. For example, the control unit 60 may control the water outlet sterilization module 80 based on the water quality measurement results of purified water.

The control unit 60 may be connected to each component provided in the water dispensing apparatus. For example, the control unit 60 can transmit and/or receive signals between each component provided in the water dispensing apparatus and control the overall operation of each Component.

The control unit 60 may include at least one processor, and may control the overall operation of the water dispensing apparatus using the processor included therein. Herein, the processor may be a general processor such as a central processing unit (CPU). The processor may be a dedicated device such as an ASIC, or may be any of other hardware-based processors.

The control unit 60 may perform various calculations based on data received through the water quality measuring unit 50 including various sensors such as the turbidity sensor 51. In addition, the control unit 60 may store data received through the water quality measuring unit 50 in a memory (not shown).

The water quality measuring unit 50 may measure water quality and output the same to the control unit 60. The control unit 60 may be controlled to perform a feedback operation in response to water quality measurement data of raw water and/or purified water. Alternatively, the water quality measuring unit 50 may directly discriminate a contamination level and transmit the same to the control unit 60, and the control unit 60 may control other components to perform appropriate feedback operations based on the received contamination level.

The control unit 60 may control the output unit 85 to recognize the contamination state of raw water and/or purified water and provide a user with information on a cleaning alarm or filter replacement cycle.

In addition, the control unit 60 may sense in advance any odor that may occur in a water tank 10 depending on the contamination level of raw water and/or purified water, and operate the automatic washing/sterilization logic through the sterilization module 70 and the water outlet sterilization module 80 before the customer feels so. Accordingly, the convenience and hygiene of use for non-professional users may be improved.

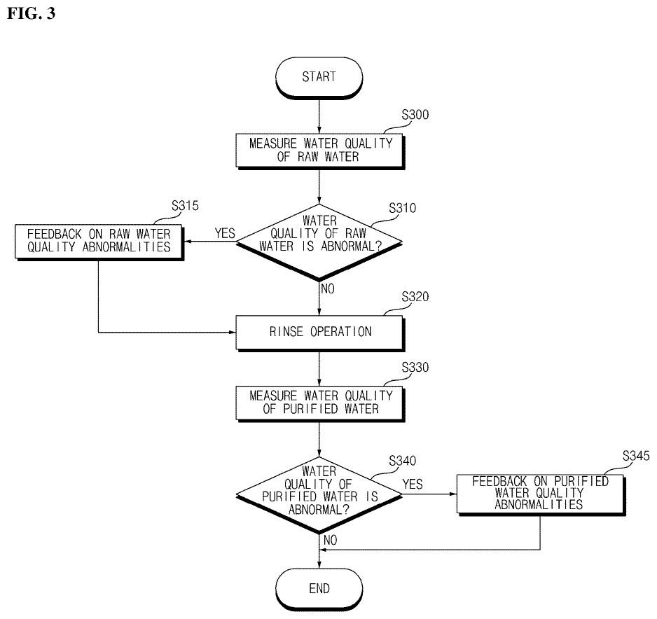

FIG. 3 is a flowchart of an operation method of a water dispensing apparatus according to an embodiment of the present disclosure.

Referring to FIG. 3, the control unit 60 supplies raw water to the water quality measuring unit 50, and the water quality measuring unit 50 measures the water quality of the raw water (S300).

The control unit 60 may be controlled to perform a feedback operation based on the water quality data measured by the water quality measuring unit 50.

For example, at the time of measuring the water quality of raw water of the water quality measuring unit 50 (S300), when the water quality data of the discriminated raw water is outside the preset normal range (S310), the control unit 60 may control the output unit 85 to display raw water quality abnormality information (S315).

When the water quality data of the raw water is not outside the preset normal range (S310), the control unit 60 may control to perform a rinse operation in which the purified water passes through the water quality measuring unit 50 at least once (S320).

Thereafter, the control unit 60 supplies raw water to the filter 10, and the water quality measuring unit 50 measures the water quality of purified water purified in the filter 10 (S330). By measuring the water quality of the purified water (S330) after the rinse operation (S320), the influence of raw water may be minimized.

When the water quality data of the discriminated purified water is outside the preset normal range (S340), the control unit 60 may control the output unit 85 to display purified water quality abnormality information (S345).

According to an embodiment of the present disclosure, a user may be provided with the raw water quality abnormality information and the purified water quality abnormality information separately, so that when there is an abnormality in purified water, the user may more quickly determine where the problem is.

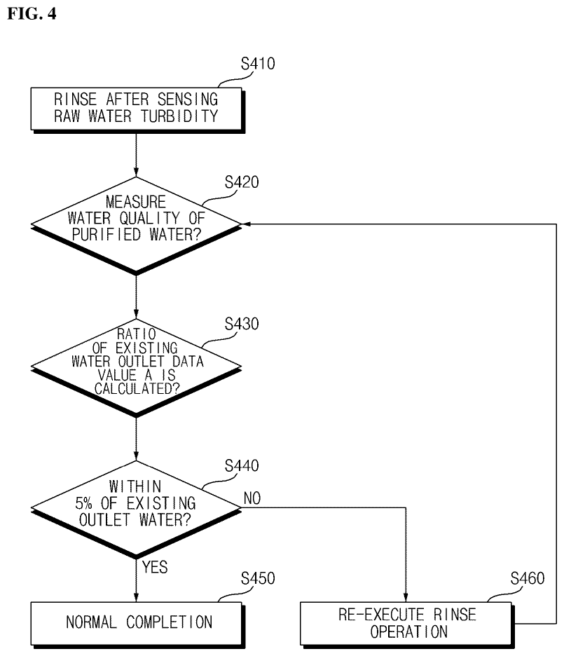

FIG. 4 is a flowchart of an operation method of a water dispensing apparatus according to an embodiment of the present disclosure.

Referring to FIG. 4, the control unit 60 may control to perform a rinse operation after measuring the water quality of raw water (S410). Thereafter, the control unit 60 may control the water quality measuring unit 50 located at a front end of the water outlet 90a to measure the water quality of purified water that may be released (S420).

The control unit 60 may compare the water quality data of purified water measured (S420) after the rinse operation (S410) with the water quality data of purified water measured before the rinse operation (S410) (S430). The control unit 60 may evaluate the difference in values of water quality data or evaluate the same as a ratio.

For example, the control unit 60 calculates (S430) the ratio of the existing water outlet data value A measured before the rinse operation (S410) and the data value measured (S420) after the rinse operation (S410). When the ratio is within a reference value (for example, 5%) compared to the existing water outlet (S440), the control unit 60 may complete the water quality measurement process normally (S450), and if not (S440), may re-execute the rinse operation (S460).

Alternatively, the control unit 60 may control the rinse operation to be performed again and the water quality of the purified water to be measured again when the difference between the existing water outlet data value A measured before the rinse operation (S410) and the data value measured (S420) after the rinse operation (S410) is greater than or equal to a predetermined reference value.

According to an embodiment of the present disclosure, by performing a rinse operation after sensing the water quality of raw water, the influence of raw water on the purified water flow path and the water quality measuring unit 50 that measures water quality in the purified water flow path may be removed, and the influence of raw water may be reconfirmed through water quality sensing at the water outlet.

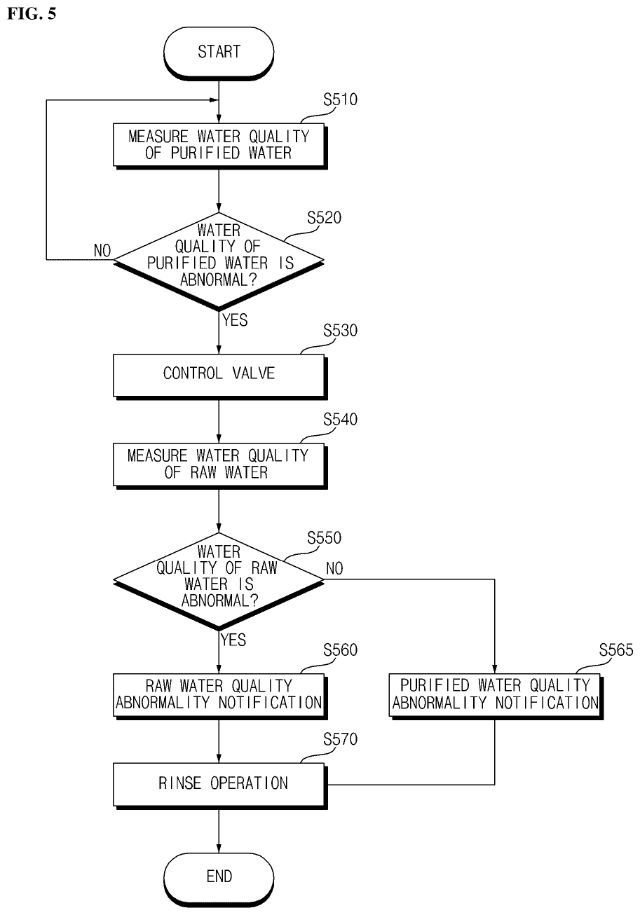

FIG. 5 is a flowchart of an operation method of a water dispensing apparatus according to an embodiment of the present disclosure.

Referring to FIG. 5, the control unit 60 supplies raw water to the filter 10, and the water quality measuring unit 50 measures the water quality of purified water purified in the filter 10 (S510).

When there is an abnormality in the water quality data of the purified water (S520), the control unit 60 may control the valve unit 90 to supply raw water to the water quality measuring unit 50 (S530).

The water quality measuring unit 50 measures the water quality of raw water (S540).

When there is an abnormality in the water quality data of the raw water (S550), the abnormality in the water quality data of the purified water (S520) is due to an abnormality in the raw water, so the control unit 60 may control the output unit 85 to output raw water quality abnormality notification information (S560).

When there is no abnormality in the water quality data of the raw water (S550), the purified water quality is lower than usual, so the control unit 60 may control the output unit 85 to output purified water quality abnormality notification information (S565).

In addition, the control unit 60 may control to perform a rinse operation after measuring the water quality of raw water (S540) (S570).

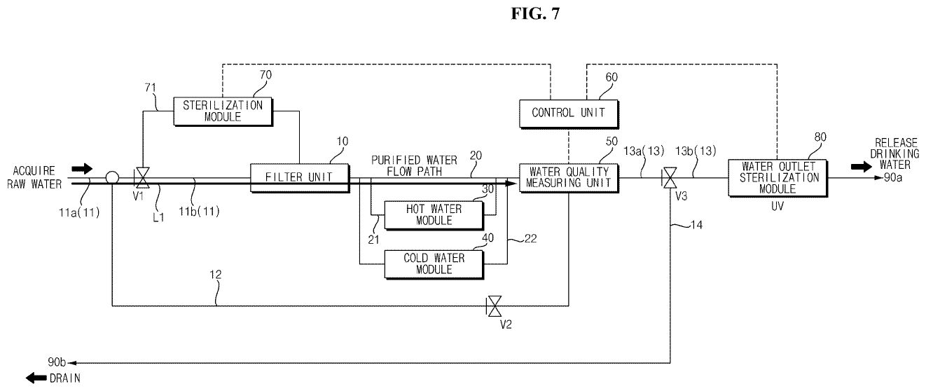

FIG. 6 is a conceptual diagram illustrating a water dispensing apparatus according to an embodiment of the present disclosure. FIGS. 7 to 9 are diagrams for reference in describing the operation of the water dispensing apparatus of FIG. 6.

Referring to FIG. 6, the water dispensing apparatus includes the water supply flow path 11 through which raw water supplied from the water supply source flows, and the filter unit 10 that filters the raw water supplied to the water supply flow path 11 to generate purified water.

The purified water that has passed through the filter unit 10 flows through the purified water flow path 20 toward the water outlet 90a. The purified water that has passed through the filter unit 10 may enters the water quality measuring unit 50. The water quality measuring unit 50 may measure the water quality of purified water when the purified water enters.

In addition, the sensing flow path 12 is branched off from the water supply flow path 11, so that raw water may directly enter the water quality measuring unit 50 through the sensing flow path 12. The water quality measuring unit 50 may measure the water quality of raw water when the raw water enters.

According to an embodiment of the present disclosure, a water supply valve V1 that controls the water supply to the filter unit 10 and the purified water flow path 20 may be disposed in the water supply flow path 11. The water supply valve V1 may open and close the purified water flow path 20. When the water supply valve V1 is opened, raw water may be purified through the filter unit 10 along a first line L1 of FIG. 7, and purified water may enter the water quality measuring unit 50 through the purified water flow path 20.

The water supply flow path 11 may include a first water supply flow path 11a connecting the water supply source and the water supply valve V1, and a second water supply flow path 11b connecting the water supply valve V1 and the filter 10.

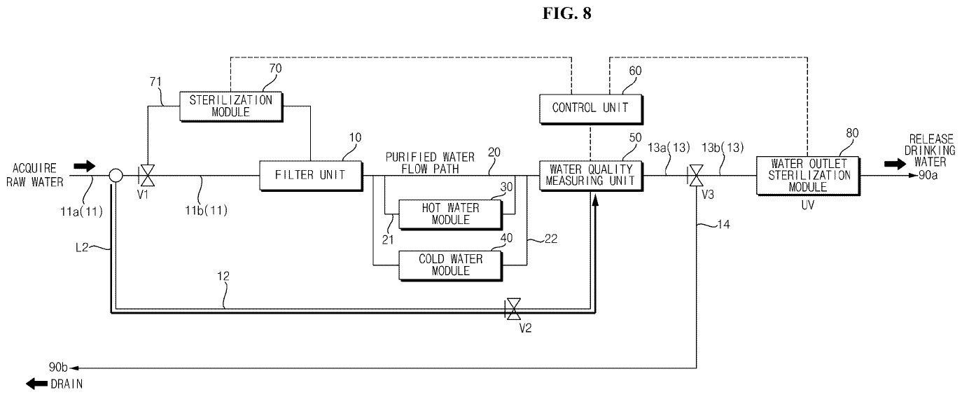

In addition, one end of the sensing flow path 12 may be connected to the first water supply flow path 11a, and the other end thereof may be connected to the water quality measuring unit 50. A sensing valve V2 that opens and closes the sensing flow path 12 may be disposed in the sensing flow path 12. When the sensing valve V2 is opened, raw water may directly enter the water quality measuring unit 50 through the sensing flow path 12 along a second line L2 of FIG. 8.

Referring to FIG. 6, the water dispensing apparatus may further include: the water outlet 90a through which the purified water is discharged; a water outlet flow path 13 guiding the purified water to the water outlet 90a; the drainage flow path 14 branched off from the water outlet flow path 13 between the water quality measuring unit 50 and the water outlet 90a through which the raw water or the purified water is drained; and a water outlet valve V3 selectively supplying the raw water or the purified water to the water outlet flow path 13 and the drainage flow path 14.

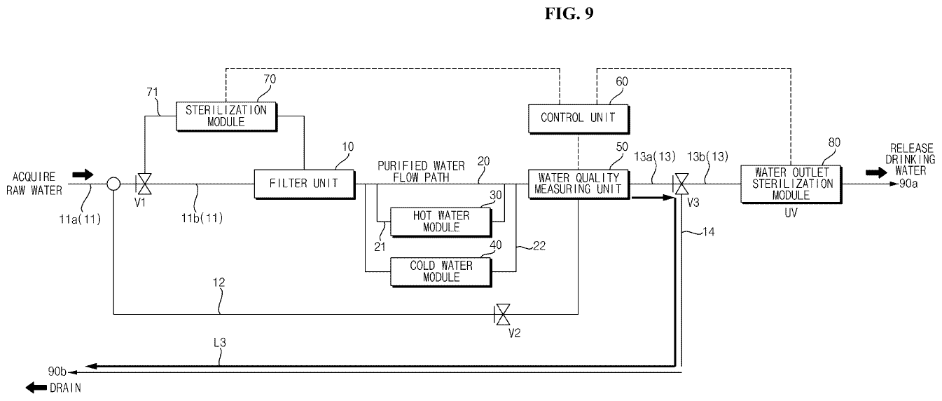

The water outlet valve V3 may, under the control of the control unit 60, divert water for which water quality measurement is completed to the drain 90b and the water outlet 90a. When the drainage operation is performed, the water for which water quality measurement is completed is allowed to flow in the drainage flow path 14 along a third line L3 of FIG. 9. By draining and washing raw water, the raw water may be prevented from being released as drinking water.

The water outlet flow path 13 may include a first water outlet flow path 13a connecting the water quality measuring unit 50 and the water outlet valve V3, and a second water outlet flow path 13b connecting the water outlet valve V3 and the water outlet 90a.

On the side of the water outlet 90a through which the purified water is discharged, the water outlet sterilization module 80 that irradiates ultraviolet rays to the water outlet 90a may be disposed. The water outlet sterilization module 80 may sterilize a water outlet space and residual water. The control unit 60 may operate the water outlet sterilization module 80 for a predetermined time based on the water quality data measured by the water quality measuring unit 50.

Referring to FIG. 6, a sterilization flow path 71 has one side branched off from the water supply flow path 11 and the other side connected to the filter 10, and the sterilization module 70 that heats water passing through the sterilization flow path 71 may be disposed in the sterilization flow path 71. The control unit 60 may operate the sterilization module 70 for a predetermined time based on the water quality data measured by the water quality measuring unit 50.

According to an embodiment of the present disclosure, when the sterilization flow path 71 is provided, the water supply valve V1 may be a switching valve that selectively supplies the raw water to the water supply flow path 11 or the sterilization flow path 71.

Referring to FIG. 6, the water dispensing apparatus may further include: a hot water flow path 21 branched off from the purified water flow path 20 at one side; the hot water module 30 provided on the hot water flow path 21 to heat purified water passing through the hot water flow path 21; a cold water flow path 22 branched off from the purified water flow path 20 at one end side; and a cooling module 40 provided on the cold water flow path 22 to cool purified water passing through the cold water flow path 22.

Referring to FIG. 6, the hot water flow path 21 and the cold water flow path 22 may be joined again to the purified water flow path 2. Alternatively, the hot water flow path 21 and the cold water flow path 22 may be joined to the water outlet flow path 13.

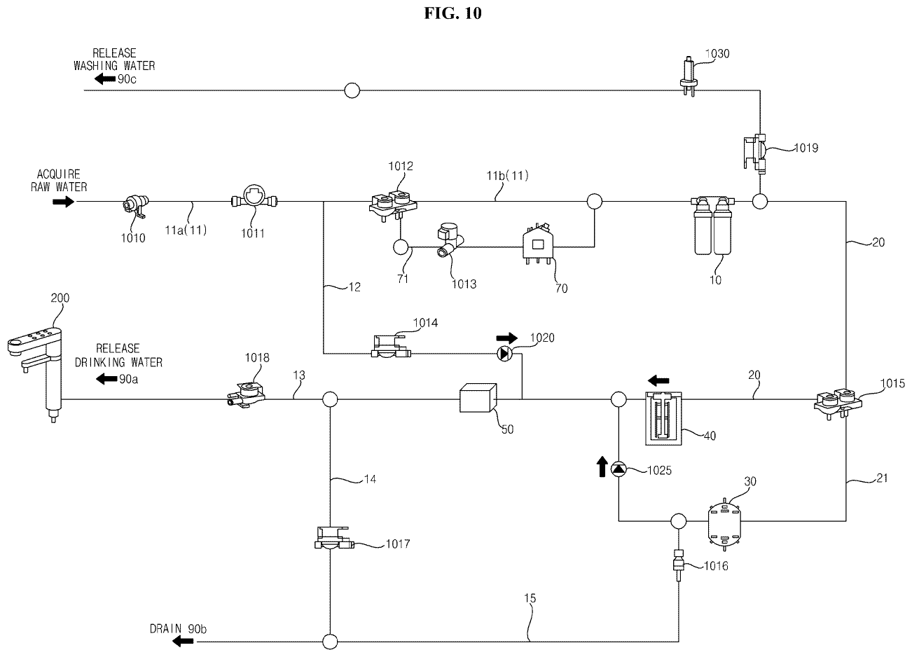

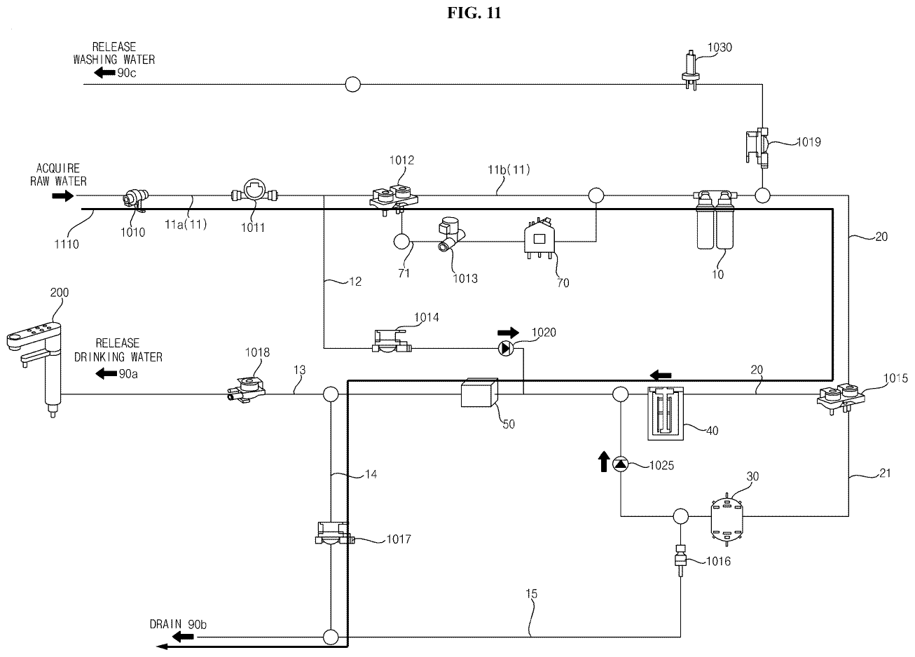

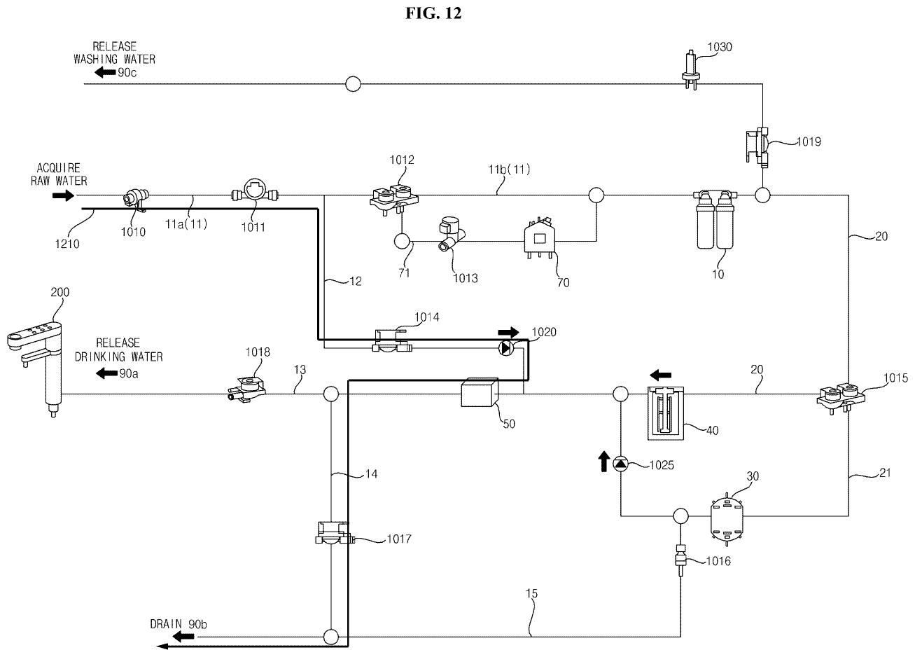

FIG. 10 is a conceptual diagram illustrating a water dispensing apparatus according to an embodiment of the present disclosure. FIG. 11 and FIG. 12 are diagrams for reference in describing the operation of the water dispensing apparatus of FIG. 10. FIG. 10 illustrates a water piping diagram of the water dispensing apparatus according to an embodiment of the present disclosure, FIG. 11 illustrates a purified water sensing and drainage line 1110, and FIG. 12 illustrates a raw water sensing and drainage line 1210.

The water dispensing apparatus according to an embodiment of the present disclosure may correspond to various water treatment apparatuses and purification apparatuses, such as water purifiers and refrigerators where water enters from the outside, purify the entered water, and then discharge the purified water.

As an example, the water dispensing apparatus may be provided with an under sink type water purifier in which at least a portion is disposed in the lower space of a sink.

Referring to FIGS. 10 to 12, the water dispensing apparatus according to an embodiment of the present disclosure may include a water outlet 200 installed so that at least a portion thereof is exposed to the outside of the sink and a remaining main body unit installed on the inside of the sink.

The water dispensing apparatus includes the water supply flow path 11 that guides raw water supplied from the outside to the inside, the filter 10 that purifies the raw water supplied along the water supply flow path 11 into purified water, and the purified water flow path 20 that causes purified water passing through the filter 10 to flow toward the water outlet 200.

The water supply flow path 11 connects an external water supply source and the filter 10. Through the water supply flow path 11, raw water supplied from the external water supply source may be supplied to the filter 10.

As described above, water (raw water) supplied to the filter 10 is purified into purified water by passing through the filter 10. At least one of the filters 10 may be provided. For example, a plurality of filters 10 may be provided. Accordingly, water passing through the water supply flow path 11 may be purified into cleaner water by passing through the plurality of filters 10.

In addition, purified water passing through the filter 10 may flow through the purified water flow path 20 toward the water outlet 200 exposed on the outside of the sink 10.

To this end, one end of the purified water flow path 20 is connected to the filter 10, and the other end is connected to the water outlet 200. In the purified water flow path 20, at least one of the cold water flow path 22, the hot water flow path 21, or a washing water flow path 90c may be branched.

In FIG. 10, the cold water flow path 22 is integrated into the purified water flow path 20, and an example is illustrated in which the hot water flow path 21 and the washing water flow path 90c are branched off from the purified water flow path 20.

One end of the purified water flow path 20 is connected to the filter 10, and water passing through the filter 10 flows toward the water outlet 200 through the connected water outlet flow path 13. The outlet 200 includes the water outlet 90a and may take out purified water.

In the purified water flow path 20, water may be cooled and supplied as cold water according to the operation of the cold water module 40. After being cooled in the cold water module 40, the purified water may be supplied to the water outlet 200 in a cold water state.

In addition, water branched into the hot water flow path 21 is heated through the hot water module 30 provided on the hot water flow path 21 and then supplied to the water outlet 200 in a hot water state.

In addition, water branched into the washing water flow path 90c may be supplied to a washing water outlet in the state of sterilized water while passing through a washing water module 1030 provided on the washing water flow path 90c. In the case where the water outlet 200 includes a plurality of water outlets, the washing water outlet may also be formed in the water outlet 200 depending on an embodiment.

A pressure reducing valve 1010 for adjusting the flow rate of water supplied to the filter 10 may be installed in the water supply flow path 11.

In addition, at least one of a flow sensor 1011 for sensing the flow rate of water, an inlet valve for adjusting the flow rate of water or regulating the flow of water, or a flow rate sensor (not shown) for sensing the flow rate of water may be installed in the water supply flow path 11 or the purified water flow path 20.

In addition, separate opening/closing valves for regulating water flow in each of the purified water flow path 20, the hot water flow path 21, and the washing water flow path 90c may be installed. For example, a washing water valve 1019 may be disposed in the washing water flow path 90c.

Alternatively, a cold/hot/purified water valve 1015 that may selectively supply purified water to the purified water flow path 20 and the hot water flow path 21 may be installed at a branch point of the purified water flow path 20 and the hot water flow path 21.

In addition, a device 1025 for safety such as backflow prevention may be installed [symbol] in the hot water flow path 21. In addition, a safety valve 1016 for steam discharge may be installed in the hot water module 30. The steam from the hot water module 30 may be drained toward the drain 90b through the connected flow path 15.

A water outlet valve 1018 may be disposed in the water outlet flow path 13 to supply or block purified water, cold water, and hot water flowing toward the water outlet 200 to the water outlet 200.

In addition, the drainage flow path 14 is branched off from the water outlet flow path 13, and a drain valve 1017 is disposed in the drainage flow path 14 so that purified water, cold water, hot water, and raw water may be discharged toward the drain 90b.

As an example, the water outlet valve 1018 and the drain valve 1017 may be provided as a three-way valve having one inlet, a first outlet and a second outlet that are selectively opened, and an actuator that selectively opens and closes the two outlets. In this connection, the first outlet may be connected to the water outlet 200, and the second outlet may be connected to the drain 90b.

Raw water is supplied through the water supply flow path 11 connected to the water supply source such as a tap water pipe, a water tank, or an underground water pipe. The pressure reducing valve 1010 is installed in the water supply flow path 11, and the raw water is reduced to a set pressure while passing through the pressure reducing valve 1010.

In addition, the raw water that passes through the filter 10 becomes purified with foreign substances removed. The purified water flows along the purified water flow path 20. In addition, the purified water may be branched into cold-purified water and hot water.

First, the purified water branched into the cold-purified water is branched again into cold water and purified water. Depending on the operation of the cold water module 40 corresponding to the purified water or cold water selection manipulation of a user, the purified water or cold water may be supplied to the user through the water outlet 200.

When a user requests the release of cold water, purified water passes through a cooling coil inside the cold water module 40. The water flowing along the cooling coil exchanges heat with the coolant inside the cold water module 40 and is cooled into cold water. To this end, the coolant is continuously cooled to maintain a set temperature. For reference, a compressor may be driven to cool the coolant. The driving of the compressor may be determined by a cold water temperature sensor provided inside the cold water module 40. Accordingly, the coolant may always maintain a set temperature, and to this end, the driving of the compressor may be adjusted. The compressor is an inverter compressor, and its frequency is adjusted in response to the required load, and its cooling capacity may be adjusted. In other words, the compressor may be driven by inverter control and cool the coolant at optimal efficiency.

When a user requests the release of hot water, the purified water may be heated to a set temperature while passing through the hot water module 30. The hot water module 30 may be heated by induction heating, and to this end, the output of the working coil included in the hot water module 30 may be adjusted. The purified water passing through the hot water module 30 may be heated to a set temperature. The heated hot water passes through the hot water module 30 and flows toward the water outlet 200.

The sterilization flow path 71 has one side branched off from the water supply flow path 11 and the other side connected to the filter 10. In the sterilization flow path 71, a sterilization module 70 that heats water passing through the sterilization flow path 71 and a flow rate adjustment valve 1013 that controls the flow rate of the sterilization flow path 71 may be disposed.

At a location where the sterilization flow path 71 is branched off from the water supply flow path 11, a feed valve 1012 that selectively supplies the raw water to the water supply flow path 11 or the sterilization flow path 71 may be disposed.

The water supply flow path 11 may include the first water supply flow path 11a connecting the water supply source and the feed valve 1012, and the second water supply flow path 11b connecting the feed valve 1012 and the filter 10.

The sensing flow path 12 described above may be branched in the first water supply flow path 11a at a front end of the feed valve 1012. A sensing valve 1014 that opens and closes the sensing flow path 12 and a backflow prevention device 1020 that prevents backflow of raw water may be disposed in the sensing flow path 12.

Referring to FIG. 12, the control unit 60 may control the feed valve 1012 to be closed and the sensing valve 1014 to be opened so that raw water is supplied to the water quality measuring unit 50 through the sensing flow path 1014.

After measuring the water quality of raw water, the control unit 60 closes the water outlet valve 1018 and opens the drain valve 1017 to discharge the raw water measured by the water quality measuring unit 50 toward the drain 90b.

Referring to FIG. 11, the control unit 60 may control the feed valve 1012 to open toward the purified water flow path 20 and the sensing valve 1014 to close so that purified water is supplied to the water quality measuring unit 50.

In addition, the control unit 60 closes the water outlet valve 1018 and opens the drain valve 1017 to perform a rinse operation by controlling the purified water passing through the water quality measuring unit 50 to be discharged toward the drain 90b.

Thereafter, the control unit 60 may measure the water quality of purified water by supplying purified water to the water quality measuring unit 50 with the same valve control. Accordingly, the influence of the raw water may be removed and the water quality of the purified water may be accurately measured in the same water quality measuring unit 50.

Hereinbefore, although preferred embodiments of the present disclosure have been illustrated and described, the present disclosure is not limited to the specific embodiments described above, and it goes without saying that persons having ordinary skills in the technical field to which the present disclosure pertains may implement the present disclosure by various modifications thereof without departing from gist of the present disclosure defined by the claims, and such modifications are not to be construed individually from the technical spirit and scope of the present disclosure.

1. A water dispensing apparatus, comprising:
a water supply flow path having flowing therein raw water supplied from a water supply source;
a filter for generating purified water by filtering the raw water supplied to the water supply flow path;
a purified water flow path having flowing therein the purified water which has passed through the filter;
a sensing flow path branching off from the water supply flow path and having the raw water flowing therein; and
a water quality measuring unit connected to the purified water flow path and the sensing flow path so as to have the purified water or the raw water entering therein, wherein the water quality measuring unit measures the water quality of the purified water when the purified water enters through the purified water flow path, and measures the water quality of the raw water when the raw water enters through the sensing flow path.

2. The water dispensing apparatus of claim 1, wherein, when the water quality of the raw water is measured, the water quality measuring unit measures the water quality of the purified water after performing a rinse operation in which the purified water passes through at least once.

3. The water dispensing apparatus of claim 1, further comprising:
a water supply valve for opening and closing the purified water flow path; and
a sensing valve for opening and closing the sensing flow path.

4. The water dispensing apparatus of claim 3, wherein the water supply flow path comprises:
a first water supply flow path connecting the water supply source and the water supply valve; and
a second water supply flow path connecting the water supply valve and the filter,
wherein one end of the sensing flow path is connected to the first water supply flow path, and the other end thereof is connected to the water quality measuring unit.

5. The water dispensing apparatus of claim 1, further comprising a three-way valve for selectively supplying the raw water to the purified water flow path and the sensing flow path.

6. The water dispensing apparatus of claim 1, wherein the water quality measuring unit comprises a turbidity sensor that irradiates light to the raw water or a portion of the purified water and senses turbidity based on a pattern of scattered light received.

7. The water dispensing apparatus of claim 1, wherein the water quality measuring unit comprises at least one of a turbidity sensor, an ion sensor, a chlorine sensor, a total dissolved solids (TDS) sensor, or a BOD (biochemical oxygen demand) sensor, and measures at least one of turbidity, pH, residual chlorine, TDS, or dissolved oxygen of entering water.

8. The water dispensing apparatus of claim 1, further comprising:
a water outlet through which the purified water is discharged;
a water outlet flow path guiding the purified water to the water outlet;
a drainage flow path branched off from the water outlet flow path between the water quality measuring unit and the water outlet through which the raw water or the purified water is drained; and
a water outlet valve selectively supplying the raw water or the purified water to the water outlet flow path and the drainage flow path.

9. The water dispensing apparatus of claim 8, further comprising:
a hot water flow path branched off from the purified water flow path at one side;
a hot water module provided in the hot water flow path to heat purified water passing through the hot water flow path;
a cold water flow path branched off from the purified water flow path at one side; and
a cooling module provided in the cold water flow path to cool purified water passing through the cold water flow path.

10. The water dispensing apparatus of claim 9, wherein the hot water flow path and the cold water flow path are joined to the water outlet flow path or joined again to the purified water flow path.

11. The water dispensing apparatus of claim 9, further comprising a water outlet sterilization module that irradiates ultraviolet rays to the water outlet.

12. The water dispensing apparatus of claim 1, further comprising:
a sterilization flow path having one side branched off from the water supply flow path and the other side connected to the filter; and
a sterilization module provided in the sterilization flow path to heat water passing through the sterilization flow path.

13. The water dispensing apparatus of claim 12, further comprising:
a switching valve for supplying the raw water to the water supply flow path or the sterilization flow path; and
a sensing valve for opening and closing the sensing flow path.

14. The water dispensing apparatus of claim 1, further comprising a control unit that controls to perform a feedback operation based on water quality data measured by the water quality measuring unit.

15. The water dispensing apparatus of claim 14, wherein, when the water quality data of the raw water is not outside a preset normal range, the control unit controls to perform a rinse operation in which the purified water passes through the water quality measuring unit at least once.

16. The water dispensing apparatus of claim 15, wherein the control unit performs the rinse operation again and measure the water quality of the purified water again when the water quality data of the purified water measured after the rinse operation differs by a reference value or more from the water quality data of the purified water measured before the rinse operation.

17. The water dispensing apparatus of claim 14, further comprising an output unit that displays raw water quality abnormality information when the water quality data of the raw water is outside a preset normal range, and displays purified water quality abnormality information when the water quality data of the purified water is outside a preset normal range.

18. The water dispensing apparatus of claim 17, wherein, when there is an abnormality in the water quality data of the purified water, after measuring the water quality of the raw water, the control unit controls the output unit to output raw water quality abnormality notification information when there is an abnormality in the water quality data of the raw water, and controls the output unit to output purified water quality abnormality notification information when there is no abnormality in the water quality data of the raw water.

19. The water dispensing apparatus of claim 14, further comprising:
a water outlet through which the purified water is discharged; and
a water outlet sterilization module that irradiates ultraviolet rays to the water outlet,
wherein the control unit operates the water outlet sterilization module for a predetermined time based on the water quality data measured by the water quality measuring unit.

20. The water dispensing apparatus of claim 14, further comprising:
a sterilization flow path having one side branched off from the water supply flow path and the other side connected to the filter; and
a sterilization module provided in the sterilization flow path to heat water passing through the sterilization flow path,
wherein the control unit operates the sterilization module for a predetermined time based on the water quality data measured by the water quality measuring unit.
